(19) United States
(12) Patent Application Publication
(10) Pub. No.: US 2013/0116581 A1
(43) Pub. Date: May 9, 2013

(54) ROLL OF PRE-CUT DISPOSABLE PROTECTIVE COVERS FOR SPHYGMOMANOMETERS

(71) Applicants: Sean D. Smith, China, ME (US); Christine A. Smith, China, MN (US); Nancy T. Lord, Pahrump, NV (US)

(72) Inventors: Sean D. Smith, China, ME (US); Christine A. Smith, China, MN (US); Nancy T. Lord, Pahrump, NV (US)

(21) Appl. No.: 13/572,614

(22) Filed: Aug. 11, 2012

Related U.S. Application Data
(63) Continuation-in-part of application No. 13/572,606, filed on Aug. 10, 2012.
(60) Provisional application No. 61/629,798, filed on Nov. 28, 2011.

(51) Int. Cl. A61B 5/022

(52) U.S. Cl. CPC A61B 5/02233
USPC 600/499

(57) ABSTRACT

This invention relates to a roll of pre-cut disposable protective covers for blood pressure cuffs provided in a continuous web of pre-cut disposable protective covers for sphygmomanometers formed from one inner layer of water impermeable material and another layer of absorbent material, and methods of preventing the spread of infection by the use of the disposable protective covers.

### CROSS REFERENCE

This application claims priority as a Continuation in Part of Non-Provisional Application 13/572,606 filed on Aug. 10, 2012, which claims priority of Provisional Application No. 61/629,798 filed on Nov. 28, 2011.

### FEDERALLY SPONSORED RESEARCH OR DEVELOPMENT

None.

### FIELD OF INVENTION

The present invention relates to a roll of pre-cut disposable protective covers for sphygmomanometers that are torn from the roll and removably secured to a sphygmomanometer capable of protecting the cuff from becoming contaminated and/or contaminating the patient.

### BACKGROUND OF INVENTION

The use of disposable covers for sphygmomanometers, is known in the prior art. More specifically, disposable covers of know designs and configurations previously devised and utilized for the purpose of protecting cuffs from becoming contaminated are known to consist basically of familiar, expected, and obvious structural configurations, notwithstanding the myriad of designs encompassed by the crowded prior art which has been developed for the fulfillment of countless objectives and requirements.

By way of example, U.S. Pat. No. 6,364,843, to Lightle discloses a medical sanitation system that allows a disposable cover to be removably secured to a sphygmomanometer and for protecting the cuff from being contaminated or from contaminating the patient. U.S. Pat. No. 5,228,448 to Byrd discloses a protective cover for a sphygmomanometer. U.S. Pat. No. 5,513,643 to Suite discloses a disposable protection wrap for use with a sphygmomanometer. Lastly, U.S. Pat. No. 5,620,002 to Byre et al discloses a universal sphygmomanometer cover.

While these devices fulfill their respective, particular objectives and requirements, the aforementioned patents do not describe a roll of pre-cut disposable protective covers for sphygmomanometers that allows a disposable cover to be removably secured to a sphygmomanometer and for protecting the cuff from being contaminated or from contaminating the patient. In this regard, the present invention substantially fulfills this need.

### SUMMARY OF INVENTION

In view of the foregoing disadvantages inherent in the known types of disposable covers of known designs and configurations now present in the prior art, the present invention provides an improved medical sanitation system. As such, the general purpose of the present invention, which will be described subsequently in greater detail, is to provide a pre-and method which has all the advantages of the prior art and none of the disadvantages.

This inventor has discovered a pre-cut disposable protective cover for sphygmomanometers that may be provided in a roll that may be portable and easily dispensed by the user to stop infectious disease from being spread from one person to another after the use of the sphygmomanometer. This is similar to the precautions regular taken by dentists on the head rests of their dental head rests, and that of the public using toilet seat coverings at public rest stops and bathrooms. The disposable protective cover is to be placed on one's arm prior to the use of the sphygmomanometer. In sweaty locker rooms, when pores are open, prisons, third world countries, the danger of infection will be reduced by disposing of the protective cover after each use and using a new one on the next person, readily available from the roll. This will shield the next person from the exposure to germs from someone else who may have had open sores, cuts, infections, open pores, and the like.

The disposable protective covers for sphygmomanometers of this invention are designed to be placed under the sphygmomanometer for sanitary purposes. This will prevent the spread of infection by placing a barrier between the skin and the sphygmomanometer thus avoiding direct contact and is particularly important in light of the increase in bacterial invections such as Methicillin-resistant Staphylococcus aureus (MRSA) that can be transmitted through even a small sore on the arm where the sphygmomanometer is usually placed.

This disposable protective cover has a non-permeable coating such as thin plastic, similar to a plastic bag, on the outside, and an inner absorbent layer such as moleskin or a similar material which will be in direct contact with the skin to absorb any leakage from a wound or skin abrasion. The size of the disposable protective cover is about seven inches wide by sixteen inches long so as to accommodate a standard size sphygmomanometer, but other sizes can be produced to accommodate pediatric and obese patients. In one embodiment, one end of the cover has a sticky strip to close the cover around the arm. This protective cover will comply with the Infection Control Standards by preventing the spread of any fluids from one person to another and will be discarded in the hazardous waste with disposable gloves, bandages, and the like. This will comply with hospitals' Hazardous Waste Disposal Standards.

The disposable protective cover is provided in a roll of pre-cut disposable protective covers for sphygmomanometers present as a continuous web of pre-cut disposable protective covers for sphygmomanometers formed from one layer of water impermeable material and another layer of absorbent material, each of said cuffs comprising: a front layer, said front layer having an inner surface, an outer surface, first and second side edges, a top edge and a bottom edge; a inner layer, said inner layer having an inner surface, an outer surface, first and second side edges, a top edge and a bottom edge; said front layer being joined to said inner layer at respective first and second side edges thereof; said front layer being sealed to said inner layer adjacent respective bottom edges thereof; an open mouth disposed at said top edges; said front and inner layers of each of said cuffs being removably attached by a front straight perforation line at said bottom edge to said top edge of the front layer of a subsequent bag in said web and a inner straight perforation line at said bottom edge to said top edge of the inner layer of a subsequent bag in said web, said front straight perforation line having a front first end and a front second end; said inner straight perforation line having a inner first end and a inner second end; said front and inner straight perforation lines being in registration with each other; and said web being rolled to form a roll of pre-cut disposable protective covers for sphygmomanometers.

The standard size for a pre-cut disposable protective cover for sphygmomanometers is a rectangular configuration with long parallel upper and lower edges about 16 inches in length and with short parallel end edges about 7 inches in height, and with a thickness of about 1/16 inch, for preventing body fluids from contacting and contaminating the sphygmomanometer between uses on successive patients. Disposable protective covers can be made available in smaller sizes for pediatric patients and larger sizes for obese patients.

The disposable protective covers for sphygmomanometers have one layer that is a water permeable material such as plastic, and said one layer of absorbent material such as a gauze of moleskin. Each of the disposable protective covers for sphygmomanometers may be further comprised of sticky tape at the top or bottom edge, which in a specific embodiment is about ¾ inch wide and extends the across the full width at the top or bottom edge. The roll of pre-cut disposable protective covers may be provided in a suitable dispenser, specifically one that may be carried by the health care professional. Suitable dispensers are found in the prior art, for instance, U.S. Pat. No. 5,975,394, to Shuvall, teaches a portable bag dispenser and U.S. Pat. No. 5,167,377, to Chalmers, teaches a portable dispenser for animal waste that may be adopted for the roll of pre-cut disposable protective covers for sphygmomanometers of this invention.

Also taught is a method of preventing the spread of infection through measuring of blood pressure in the limb of a human body by a health care professional comprising: a) obtaining a roll of pre-cut disposable protective covers for sphygmomanometers comprising: a continuous web of pre-cut disposable protective covers for sphygmomanometers formed from one layer of water impermeable material and another layer of absorbent material, each of said covers comprising: a outer layer, said outer layer having an inner surface, an outer surface, first and second side edges, a top edge and a bottom edge; a inner layer, said inner layer having an inner surface, an outer surface, first and second side edges, a top edge and a bottom edge; said outer layer being joined to said inner layer at respective first and second side edges thereof; said outer layer being sealed to said inner layer adjacent respective bottom edges thereof; an open mouth disposed at said top edges; said outer and inner layers of each of said cuffs being removably attached by a outer straight perforation line at said bottom edge to said top edge of the outer layer of a subsequent bag in said web and a inner straight perforation line at said bottom edge to said top edge of the inner layer of a subsequent bag in said web, said outer straight perforation line having a outer first end and a outer second end; said inner straight perforation line having a inner first end and a inner second end; said outer and inner straight perforation lines being in registration with each other; and said web being rolled to form a roll of pre-cut disposable protective covers for sphygmomanometers; b) tearing one pre-cut disposable protective cover for a sphygmomanometer; c) wrapping said pre-cut disposable protective cover for a sphygmomanometer around the patients limb such that the one water impermeable layer is the inner layer directly in contact with the patient's limb and the one absorbent layer is the outer layer to be directly in contact with a strap of a standard sphygmomanometer; d) applying a standard sphygmomanometer around the patient's limb over the pre-cut disposable protective cover; e) measuring the patient's blood pressure with said standard sphygmomanometer; f) removing said sphygmomanometer and pre-cut disposable protective cover; g) disposing of said pre-cut disposable protective cover in accordance with Hazardous Waste Disposal Standards; and h) repeating steps a) - g) on the next patient, if any.

The disposable protective covers used in the method of preventing the spread of infection may be further comprised an adhesive of the type on catamenial shields attachable to panties on the inner water impermeable surface of the top or bottom edge of the disposable protective cover adapted to be coupled to the exterior surface of said disposable cover after said cover is wrapped around a patient's limb.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a view of a single pre-cut disposable cover for sphygmomanometers observed laid flat with the interior surface on top and the adhesive tape on either the top or bottom edge.

FIG. 2 is a perspective illustration of the pre-cut disposable cover for sphygmomanometers in accordance with the principles of the present invention, observed from the top or bottom edge.

FIG. 3 is a perspective illustration of the sphygmomanometer with the disposable liner that embodies principles of the invention in a preferred form, observed from the side edge.

FIG. 4 is a perspective illustration of a single pre-cut disposable cover for sphygmomanometers observed partially rolled, with the interior surface on outside of the roll and the adhesive tape at the top edge.

FIG. 5 is an illustration of the roll of pre-cut disposable cover for sphygmomanometers.

FIG. 6 is an illustration of a single pre-cut disposable cover for sphygmomanometers wrapped around a patient's arm.

FIG. 7 is an illustration of a single pre-cut disposable cover for sphygmomanometers wrapped around a patient's arm with a sphygmomanometer applied externally to it.

### DETAILED DESCRIPTION OF THE DRAWINGS

The disposable protective cover is provided in a roll (1) of pre-cut disposable protective covers (2) for sphygmomanometers (24) present as a continuous web (3) of pre-cut disposable protective covers for sphygmomanometers formed from one outer layer of water impermeable material (4) and another inner layer of absorbent material (5) , each of said covers comprising: an outer layer (6), said outer layer having an inner surface (7), an outer surface (8), first (9) and second (10) side edges, a top (11) edge and a bottom (12) edge; an outer (13) layer, said inner layer (14) having an inner surface (15), an outer surface (16), first (17) and second (18) side edges, a top edge (19) and a bottom edge (20); said outer layer (6) being joined to said inner layer (14) at respective first (9) and second (10) side edges thereof; said outer layer (6) being sealed to said outer (13) layer at its adjacent respective bottom edges (12) thereof; an open mouth disposed at said top edges (11); said outer (6) and inner (14) layers of each of said covers (2) being removably attached by a outer straight perforation line (21) at said bottom edge (20) to said top edge (19) of the outer layer (6) of a subsequent bag (22) in said web (3) and an inner straight perforation line (23) at said bottom edge (20) to said top edge (19) of the inner layer (14) of a subsequent bag (22) in said web (3), said outer (21) and inner (23) straight perforation lines being in registration with each other; and said web (3) being rolled to form a roll (1) of pre-cut disposable protective covers (2) for sphygmomanometers (24).

The standard size for a pre-cut disposable protective cover for sphygmomanometers (24) is a rectangular configuration with long parallel side edges (17,18) about 16 inches in length and with short parallel end edges(19, 20) about 7 inches in height, and with a thickness of about 1/16 inch, for preventing body fluids from contacting and contaminating the sphygmomanometer between uses on successive patients. Disposable protective covers (2) can be made available in smaller sizes for pediatric patients and larger sizes for obese patients.

The disposable protective covers for sphygmomanometers have one layer that is a water permeable material such as plastic (4), and said one layer of absorbent material (5) such as a gauze of moleskin. Each of the disposable protective covers (2) for sphygmomanometers (24) may be further comprised of sticky tape (25) at the top (19) or bottom (20) edge, which in a specific embodiment is about ¾ inch wide and extends the across the full width at the top or bottom edge. The roll of pre-cut disposable protective covers may be provided in a suitable dispenser, specifically one that may be carried by the health care professional.

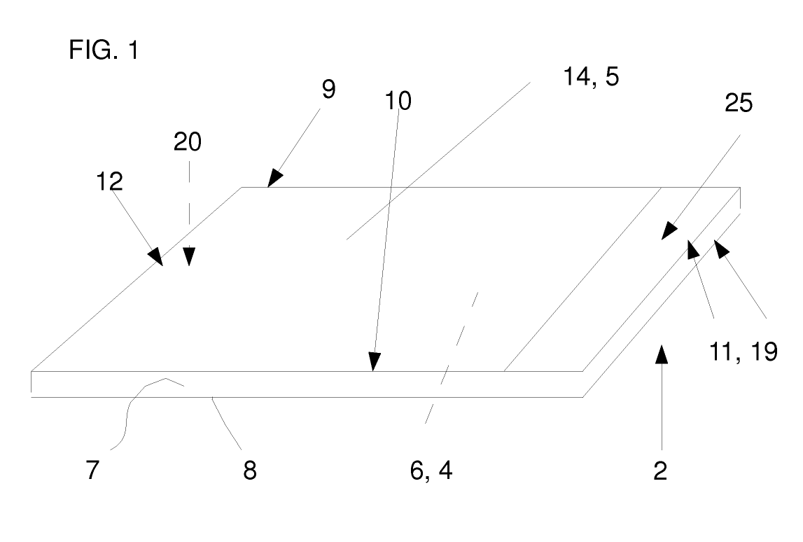

FIG. 1 is a view of a single pre-cut disposable cover (2) for sphygmomanometers [See FIG. 7] observed laid flat with the inner layer (14) over the outer layer (6) and the adhesive tape (25) on the top edge (19).

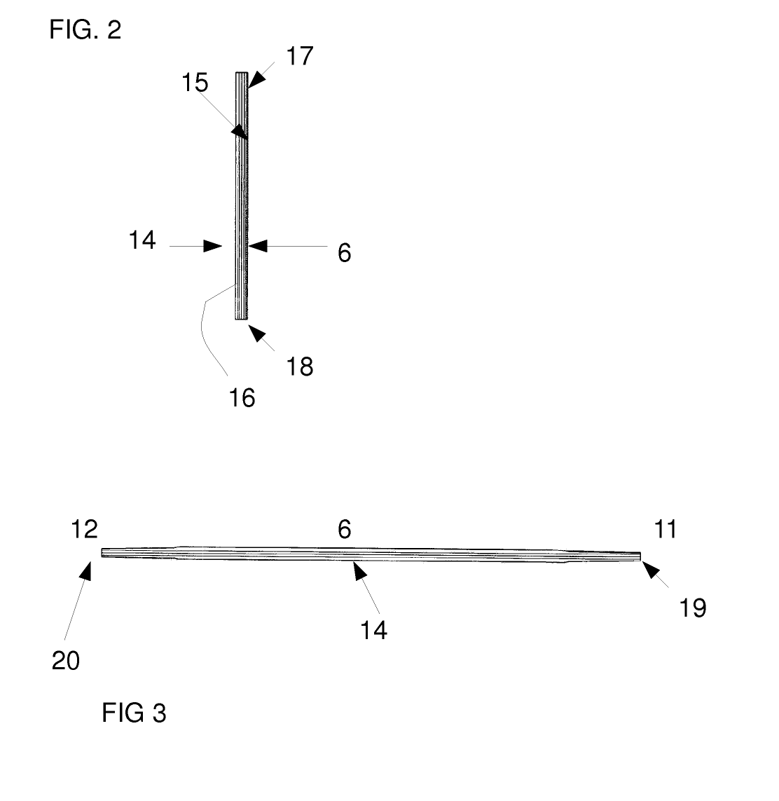

FIG. 2 is a perspective illustration of the pre-cut disposable cover (2) for sphygmomanometers [See FIG. 7] in accordance with the principles of the present invention, observed from the top (19) edge.

FIG. 3 is a perspective illustration of the pre-cut disposable cover (2) for sphygmomanometers [See FIG. 7] that embodies principles of the invention in a preferred form, observed from the side (17, 18) edge.

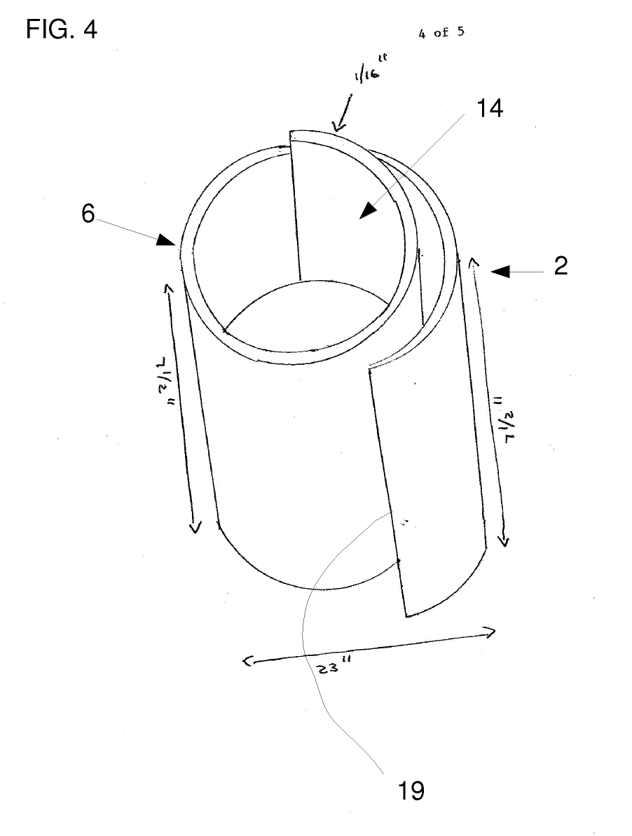

FIG. 4 is a perspective illustration of a single single pre-cut disposable cover (2) for sphygmomanometers [See FIG. 7] observed partially rolled, with the outer layer (6) on the outside of the roll and the adhesive tape (25) at the top (19) edge.

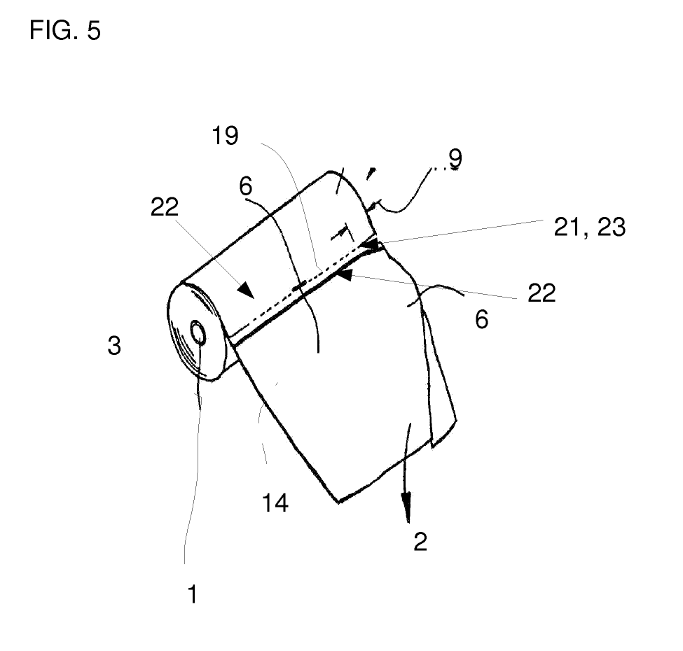

FIG. 5 is an illustration of the roll (1) of pre-cut disposable cover (2) for sphygmomanometers [See FIG. 7].

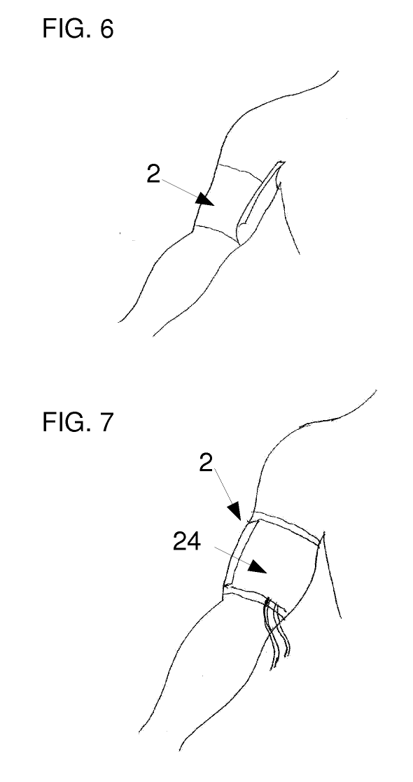

FIG. 6 is an illustration of a single pre-cut disposable cover (2) for sphygmomanometers [See FIG. 7] wrapped around a patient's arm.

FIG. 7 is an illustration of a single pre-cut disposable cover (2) for sphygmomanometers (24) wrapped around a patient's arm with a sphygmomanometer, a sphygmomanometer, (24) applied externally to it.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

1. A roll of pre-cut disposable protective covers for sphygmomanometers comprising: a continuous web of pre-cut disposable protective covers for sphygmomanometers formed from one inner layer of water impermeable material and another layer of absorbent material, each of said covers comprising: a outer layer, said outer layer having an inner surface, an outer surface, first and second side edges, a top edge and a bottom edge; a inner layer, said inner layer having an inner surface, an outer surface, first and second side edges, a top edge and a bottom edge; said outer layer being joined to said inner layer at respective first and second side edges thereof; said outer layer being sealed to said inner layer adjacent respective bottom edges thereof; an open mouth disposed at said top edges; said outer and inner layers of each of said cuffs being removably attached by a outer straight perforation line at said bottom edge to said top edge of the outer layer of a subsequent bag in said web and a inner straight perforation line at said bottom edge to said top edge of the inner layer of a subsequent bag in said web, said outer straight perforation line having a outer first end and a outer second end; said inner straight perforation line having a inner first end and a inner second end; said outer and inner straight perforation lines being in registration with each other; and said web being rolled to form a roll of pre-cut disposable protective covers for sphygmomanometers.

2. The roll of pre-cut disposable protective covers for sphygmomanometers of claim 1 wherein each of said disposable covers for a sphygmomanometer has a rectangular configuration with long parallel upper and lower edges about 16 inches in length and with short parallel end edges about 7 inches in height, and with a thickness of about 1/16 inch, for preventing body fluids from contacting and contaminating the sphygmomanometer between uses on successive patients.

3. The roll of pre-cut disposable protective covers for sphygmomanometers of claim 1 wherein said one layer of water permeable material is plastic and said one layer of absorbent material is gauze of moleskin.

4. The roll of pre-cut disposable protective covers for sphygmomanometers of claim 1 wherein each disposable protective cover is further comprised of sticky tape at the top or bottom edge.

5. The roll of pre-cut disposable protective covers for sphygmomanometers of claim 4 wherein said sticky tape is about ¾ inch wide and extends the across the full width at the top or bottom edge.

6. A method of preventing the spread of infection through measuring of blood pressure in the limb of a human body by a health care professional comprising:
a) obtaining a roll of pre-cut disposable protective covers for sphygmomanometers comprising: a continuous web of pre-cut disposable protective covers for sphygmomanometers formed from one inner layer of water impermeable material and another layer of absorbent material, each of said cuffs comprising: a outer layer, said outer layer having an inner surface, an outer surface, first and second side edges, a top edge and a bottom edge; a inner layer, said inner layer having an inner surface, an outer surface, first and second side edges, a top edge and a bottom edge; said outer layer being joined to said inner layer at respective first and second side edges thereof; said outer layer being sealed to said inner layer adjacent respective bottom edges thereof; an open mouth disposed at said top edges; said outer and inner layers of each of said cuffs being removably attached by a outer straight perforation line at said bottom edge to said top edge of the outer layer of a subsequent bag in said web and a inner straight perforation line at said bottom edge to said top edge of the inner layer of a subsequent bag in said web, said outer straight perforation line having a outer first end and a outer second end; said inner straight perforation line having a inner first end and a inner second end; said outer and inner straight perforation lines being in registration with each other; and said web being rolled to form a roll of pre-cut disposable protective covers for sphygmomanometers;
b) tearing one pre-cut disposable protective cover for a sphygmomanometer;
c) wrapping said pre-cut disposable protective cover for a sphygmomanometer around the patients limb such that the one water impermeable layer is the inner layer directly in contact with the patient's limb and the one absorbent layer is the outer layer to be directly in contact with a strap of a standard sphygmomanometer;
d) applying a standard sphygmomanometer around the patient's limb over the pre-cut disposable protective cover;
e) measuring the patient's blood pressure with said standard sphygmomanometer;
f) removing said sphygmomanometer and pre-cut disposable protective cover;
g) disposing of said pre-cut disposable protective cover in accordance with Hazardous Waste Disposal Standards;
h) repeating steps a) - g) on the next patient, if any.

7. The method as set forth in claim 6 wherein the disposable protective cover is further comprised an adhesive of the type on catamenial shields attachable to panties on the inner water impermeable surface of the top or bottom edge of the disposable protective cover adapted to be coupled to the exterior surface of said disposable cover after said cover is wrapped around a patient's limb.
